(19) United States
(12) Patent Application Publication
(10) Pub. No.: US 2011/0245871 A1
(43) Pub. Date: Oct. 6, 2011

(54) CROSSLINK ELEMENT AND BENDER FOR SPINE SURGERY PROCEDURES

(76) Inventor: Lytton A. WILLIAMS, Rolling Hills Estates, CA (US)

(21) Appl. No.: 12/755,388

(22) Filed: Apr. 6, 2010

(51) Int. Cl. A61B 17/70

(52) U.S. Cl. 606/250

(57) ABSTRACT

A crosslink element for spine surgery procedures is described. The crosslink element is made of a substantially flat shaped single piece and comprises a central region and end regions. The end regions are wider than the central region and contain slots to house hooks for connection to transversal rods in an H-construct or parallelogram spine implant. The end regions comprise insets provided on one face of the crosslink element in correspondence of borders of the slots. A bender for use with the crosslink element and a kit of parts are also described.

### FIELD

The present disclosure relates to spine surgery devices and methods. More in particular, it relates to a crosslink element and a bender for spine surgery procedures.

### SUMMARY

According to a first aspect, a substantially flat shaped single piece crosslink element for spine surgery procedures is provided, comprising a central region and end regions, the end regions being wider than the central region, each end region containing a slot adapted to house a hook, wherein the end regions comprise insets provided on one face of the crosslink element in correspondence of borders of the slots.

According to a second aspect, a hook for surgical procedures comprising a stem portion and a hook portion is provided, the hook portion adapted to be housed in a slot of the crosslink element of the first aspect above.

Further embodiments of the disclosure are shown in the description, drawings and claims of the present application.

### BRIEF DESCRIPTION OF THE DRAWINGS

FIG. 1 shows a top view of a crosslink element.

FIG. 2 shows a bottom view of the crosslink element of FIG. 1.

FIGS. 3 and 4 show side views of the crosslink element of FIGS. 1 and 2.

FIG. 5 shows an example of dorsal-ventral bending of the crosslink element.

FIG. 6 shows an example of cordal-cephalad bending of the crosslink element.

FIG. 7 shows bending of the crosslink element with one bender.

FIG. 8 shows bending of the crosslink element with two benders.

FIGS. 9 and 10 shows examples of hooks to be used in conjunction with the crosslink element.

FIGS. 11, 12 and 15 show examples of translation movement of a hook inside a slot of the crosslink element.

FIGS. 13 and 14 show examples of a first kind of rotational movement of the hook inside the slot of the crosslink element.

FIG. 16A shows an example of a second kind of rotational movement of the hook inside the slot of the crosslink element.

FIG. 16B shows an example of a third kind of rotational movement of the hook inside the slot of the crosslink element.

FIG. 17 shows an exploded view of an arrangement for engaging the hook into the slot of the crosslink element.

FIGS. 18A-18C show perspective views with two hooks engaged into respective slots of the crosslink element, where transversal rods are also shown.

FIGS. 19 and 20 show perspective views of a slit of the bender.

FIGS. 21 and 22 show perspective views of the inner section of the bottom part of the bender.

FIG. 23 shows a kit of parts containing benders, crosslink elements, and other components to be used during a spine surgery procedure.

FIG. 24 shows an example of a parallelogram structure comprising the crosslink element.

### DESCRIPTION OF EXAMPLE EMBODIMENTS

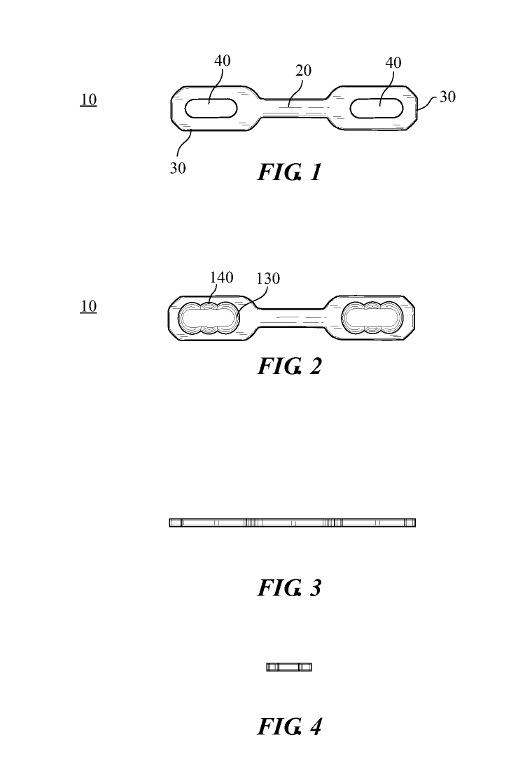

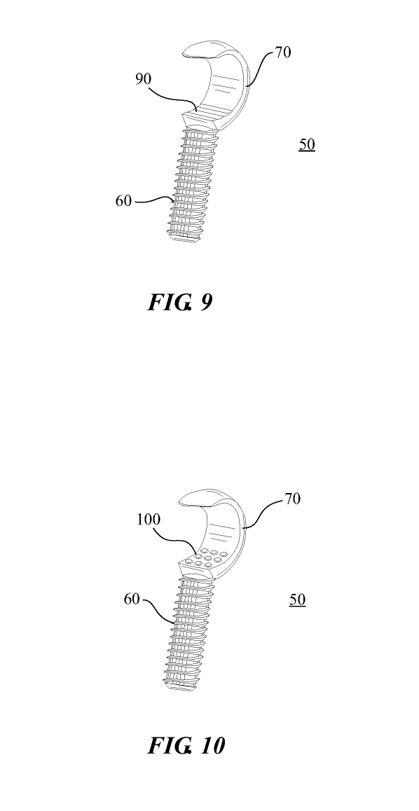

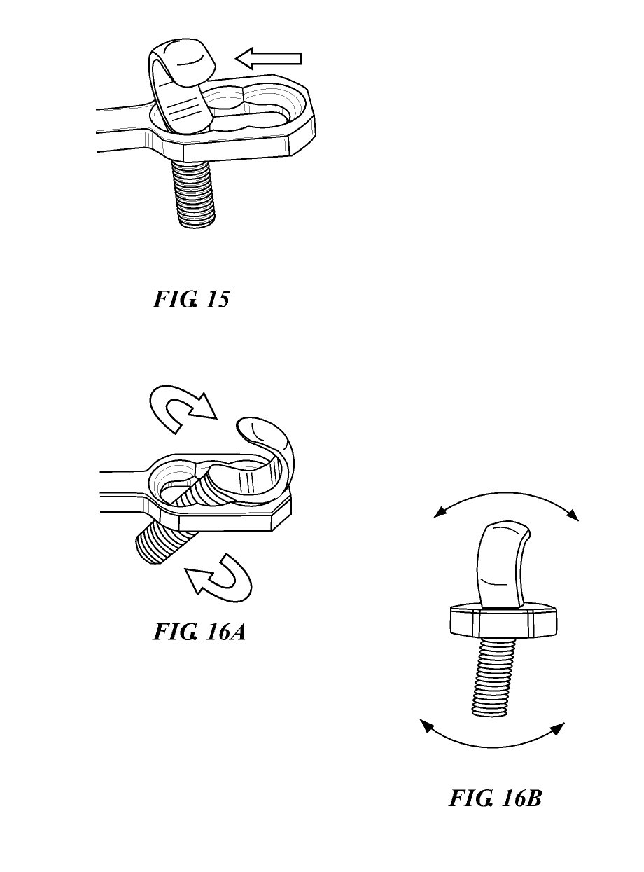

FIGS. 1-4 show a single piece crosslink element (10) having a substantially flat shape, comprised of a substantially central region (20) and end regions (30), the end regions (30) being wider than the central region. Each end region (30) of the crosslink element contains a slot (40). Each slot (40) of the crosslink element (10) is adapted to house a hook (50), shown in FIGS. 9-18 and 23. While multiple-piece crosslinks can cause dislocation and fractures in the piece, decrease its bendability and compromise the strength of the crosslink, a single piece crosslink element is bendable, increases the ease of use and decreases the amount of time a surgeon needs to implant the crosslink, thereby decreasing the overall amount of time a patient is in surgery and anesthetized.

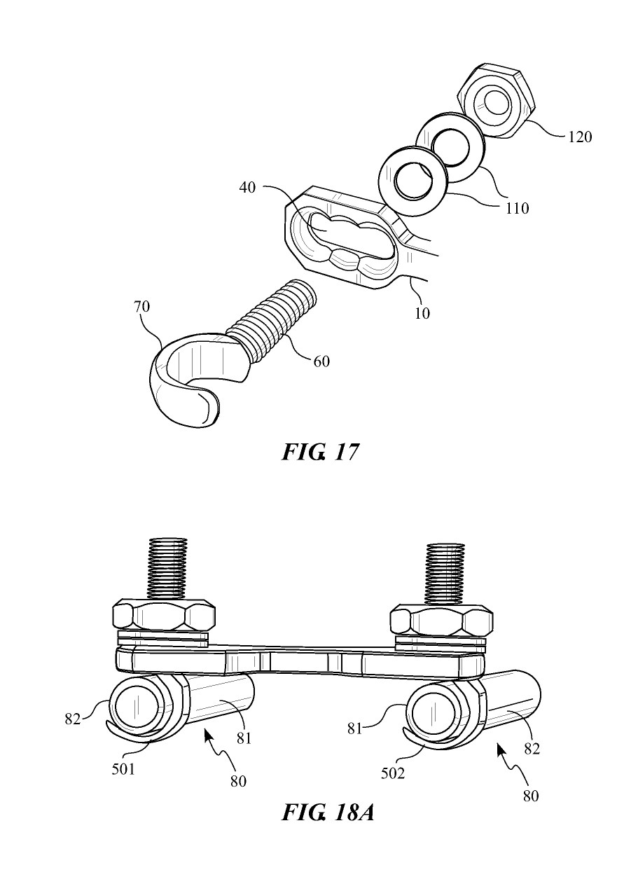

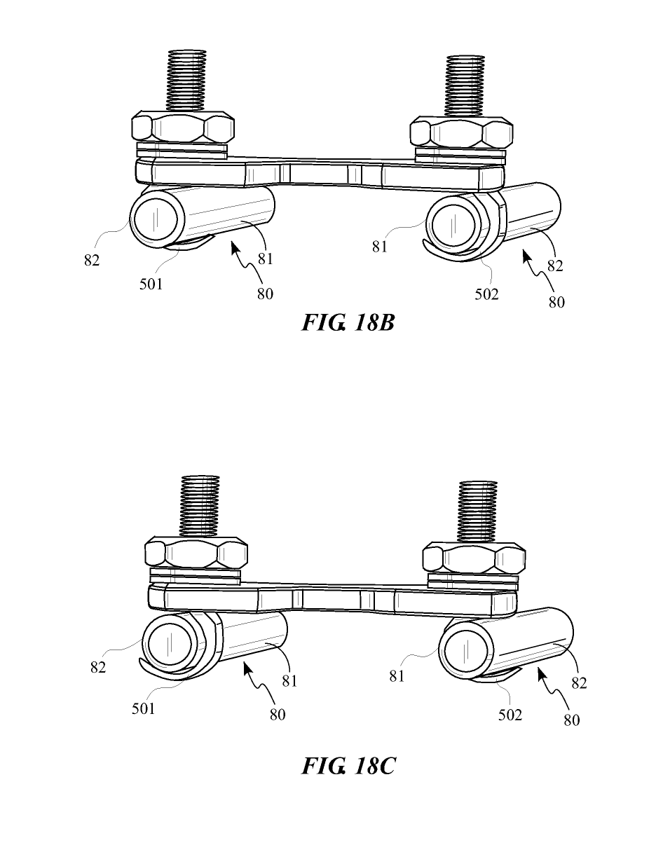

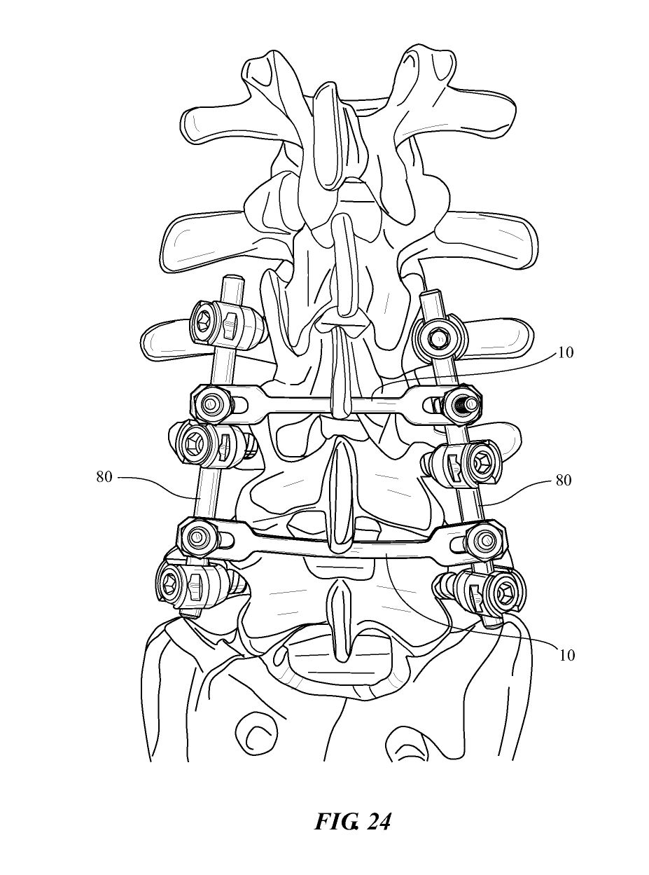

As shown, for example, in FIGS. 9 and 10, each hook (50) comprises a stem portion (60) and a hook portion (70). The hook portion (70) is adapted to be housed in a slot (40) of the crosslink element (10) of FIGS. 1-4 and engage the crosslink element (10) with a spinal rod (80), as later explained and shown, for example, in FIG. 24. The stem portion (60) of the hook (50) is adapted to stabilize, in cooperation with elements such as washers and nuts, the engagement between the crosslink element (10) and the spinal rods (80). For example, the stem portion (60) can be threaded (see, e.g., FIGS. 9 and 10) to allow rotational engagement of a nut (120) around the stem portion (60). In this respect, reference can be made to the exploded view of FIG. 17 and the perspective views of FIGS. 18A-18C, where the interplay between crosslink element (10), slot (40), stem (60), washers (110) and nut (120) is pictorially shown. Other embodiments can also be provided where, for example, the hook is fastened to the crosslink element by way of a clamp, or is integral with the crosslink element.

FIGS. 9 and 10 show embodiments of the present disclosure where the hook portion (70) can have an irregular internal surface configuration to allow a better grip between a crosslink element (10) and a rod (80). In particular, FIG. 9 shows a plurality of grooves (90) located on the internal surface of the hook portion (70), while FIG. 10 shows a plurality of beads (100). According to a further embodiment of the disclosure, the internal surface configuration can be irregular (through presence of, e.g., grooves or beads) only in correspondence of the region of the internal surface of the hook portion proximal to the stem portion (60), as also shown in FIGS. 9 and 10. In this way, the presence of the grooves or beads along the transverse/circumferential length of the transverse rods is reduced, thus reducing the chances of stress risers and fatigue fractures in the rod.

Turning to the depiction of FIG. 24, it should be noted that the attachment between spinal rods (80) and crosslink elements (10) through hooks (50) can form H-constructs or parallelograms. An H-construct is a structure comprising two rods (80) and one crosslink element (10). A parallelogram is a structure comprising two rods (80) and two crosslink elements (10). FIG. 24 shows an example of such parallelogram.

As shown in FIGS. 18A-18C, a rod (80) can have an inner side (81)—defined as the part of the rod (80) facing the crosslink element (10) when an H-construct or a parallelogram is formed—and an outer side (82)—defined as the part of the rod (80) opposite the inner side.

In this respect, a hook (50) can engage a rod (80) either on the inner side of the rod (80) or the outer side of the rod. A hook (50) engaging a rod (80) on the inner side of the rod faces towards the outside of the H-construct or parallelogram of FIG. 24 and exerts a distraction force on the crosslink element (10). On the other hand, a hook (50) engaging a rod (80) on the outer side of the rod (80) faces towards the inside of the H-construct or parallelogram and exerts a compression force on the crosslink element (10).

In particular, in accordance with the present disclosure, when two hooks (50) are placed in the slots (40) of the crosslink element (10), connection between the crosslink element (10) and the spinal rods (80) can be obtained in accordance with three different settings:
1) Both hooks engage the rods on the inner side of a respective rod. See, for example, FIG. 18C where hooks (501), (502) both engage the inner sides (81) of the rods (80).
2) Both hooks engage the rods on the outer side of a respective rod. See, for example, FIG. 18B where hooks (501), (502) both engage the outer sides (82) of the rods (80).
3) One hook engages one rod on an inner side of the rod and the other hook engages the other rod on the outer side of the rod. See, for example, FIG. 18A where hook (501) engages the inner side (81) of the left rod (80) and hook (502) engages the outer side (82) of the right rod (80).

The three different settings provide a versatile approach that helps the surgeon during implantation of the crosslink element because they provide independent distraction and/or compression capabilities for each hook. Presence of added distraction and/or compression capabilities is good because allows better stabilization and increased triangulation of the construct shown in FIG. 24, which increases the pullout strength of the fixation. In particular, the higher the compression and/or distraction force, the stronger and more stable the fixation. Moreover, the ability to fix the hook either on the inner or outer side of the rod allows for flexibility in fixation based on the anatomical presentation of a patient, such as muscle or bone blocking placement on one side of a rod. Flexibility in compression/distraction or flexibility in fixation is important because it allows the surgeon to immediately adapt to the patient's anatomical presentation, which is sometimes unpredictable before operating on the patient and exposing to the surgeon the area of interest.

As already mentioned above and shown, for example, in FIGS. 17 and 18A-18C, stability of the engagement between the crosslink element (10) and the spinal rods (80) through the hooks (50) can be obtained by inserting washers (110) and nuts (120) through the threaded stems (60) of the hooks (50).

Improved stability is also obtained by way of a particular configuration of the regions of the crosslink element surrounding the slots (40). In particular, as shown, for example, in FIGS. 2 and 6, where the crosslink element (10) is shown in a view from the bottom (FIG. 2) and in a perspective view from the bottom (FIG. 6) respectively, oval insets or depressions (130) are provided in correspondence of the borders of the slots (40). In particular, each slot (40) of the embodiments of FIGS. 2 and 6 is surrounded, around the border of its bottom surface, by three sets of oval insets. Each set of oval insets (140) comprises a plurality of curved grooves of decreasing diameter in a direction towards the slot (40) to seat the stem portion of the hook when fastened. The shape of the sets of oval insets (130) resembles a plurality of circles attached to each other and allows improved stability when the hook (50) is engaged in a particular position inside the slot (40). In the example shown in the FIGS. 2 and 6, the configuration (130) of the oval insets allows three different positions of the hook (50) when engaged in position inside the slot (40), meaning that three different positions of the connection between a hook (50) and a spinal rod (80) can be provided as soon as the hook is set into position through the crosslink element (10), embracing the rod (80), by way, for example, of a nut and washers going through the thread stem portion (60).

The possibility of providing for three different positions of a hook inside each slot allows the definition of five different distances between the hooks, thus providing the surgeon with a great flexibility when choosing the right distance between the hooks during implantation on the patient.

The three positions defined by the configuration (130) of oval insets of FIGS. 2 and 6 are shown by way of example only. Any number of positions can be provided. It can be seen that the curved configuration (130) of oval insets allows a better engagement between a hook (50) and a slot (40) because such engagement is not only determined by the strength of the coupling between a nut and the hook stem (60) but also by the engagement of the hook (50) into one of the regions (three in the examples of FIGS. 2 and 6) defined by the curved configuration (130) of oval insets, similarly to a snapping engagement.

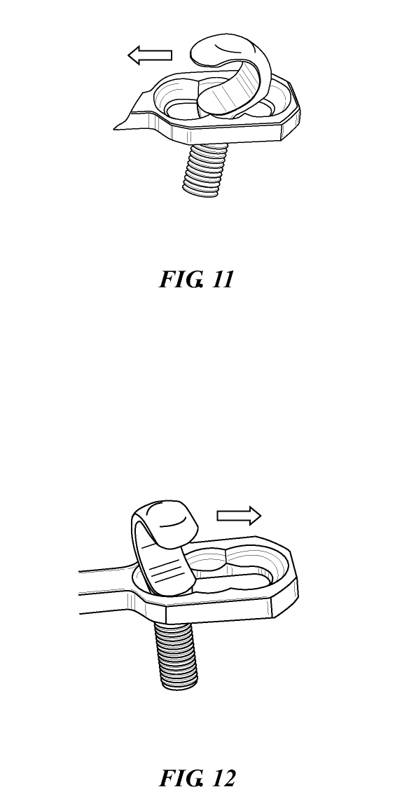

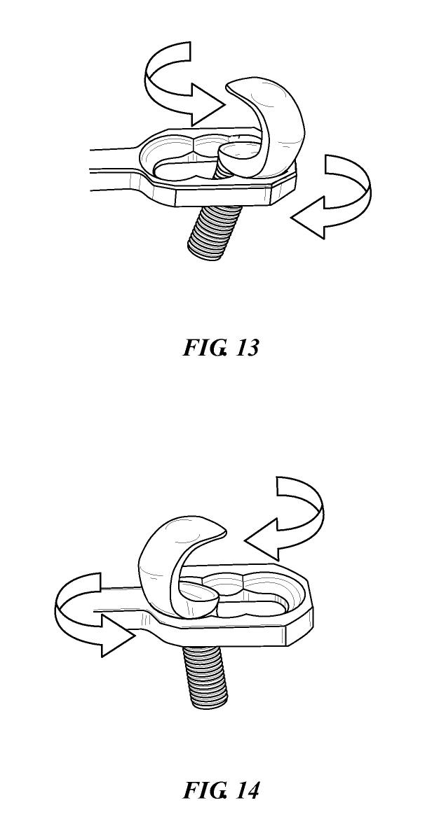

Improved freedom for the spinal surgeon during the surgical procedure is also obtained by providing a slot/hook coupling where the hook (50), before being engaged by way of a nut, has four degrees of freedom. A first degree of freedom is a translational movement across the transversal extension of the slot (40), as shown in FIGS. 11 and 12. A second degree of freedom is a 360 degree rotational movement (yaw) across the (vertical) axis of the stem (60), as shown in FIGS. 13 and 14. A third degree of freedom is a rotational movement across a transversal axis which defines a partial lateral 180 degree movement of the hook (pitch) away from and towards the crosslink element (10), as shown in FIG. 16B. A fourth degree of freedom is a rotational movement across a normal axis away from and towards the center of the crosslink element (10) (roll) in the same transversal direction of the crosslink element (10), as shown in FIG. 16A.

The four degrees of movement described above will allow the surgeon to have a large freedom of movement when implanting the crosslink element (10) on a patient. For example, configurations where the spinal rods (80) shown in FIG. 24 are at a level that is different from the level of the crosslink element (10) will be easily dealt with by the way engagement of the hook (50) is obtained in accordance with the disclosure, thus increasing the versatility of the hook when adapting to the construct of the spine. The combination of this feature with the previous feature of compression/distraction mentioned above allows a greater flexibility by which the problems due to the different anatomical presentations of the patients (due to bone and/or muscle inhibition) can be solved.

The person skilled in the art will also understand that the freedom of hook movement due to the various rotational degrees of movement of the hook inside the slot combined with the presence of the above discussed insets will allow both a “coarse” and a “fine” tuning of the distance between the hooks, therefore allowing the use of the crosslink element for any kind of distance required. In particular, the hooks can be initially placed along one of the insets to obtain an initial (coarse) placement and then rotated to better fine tune the initial placement and obtain the distance desired by the surgeon in accordance with the needs of the specific procedure.

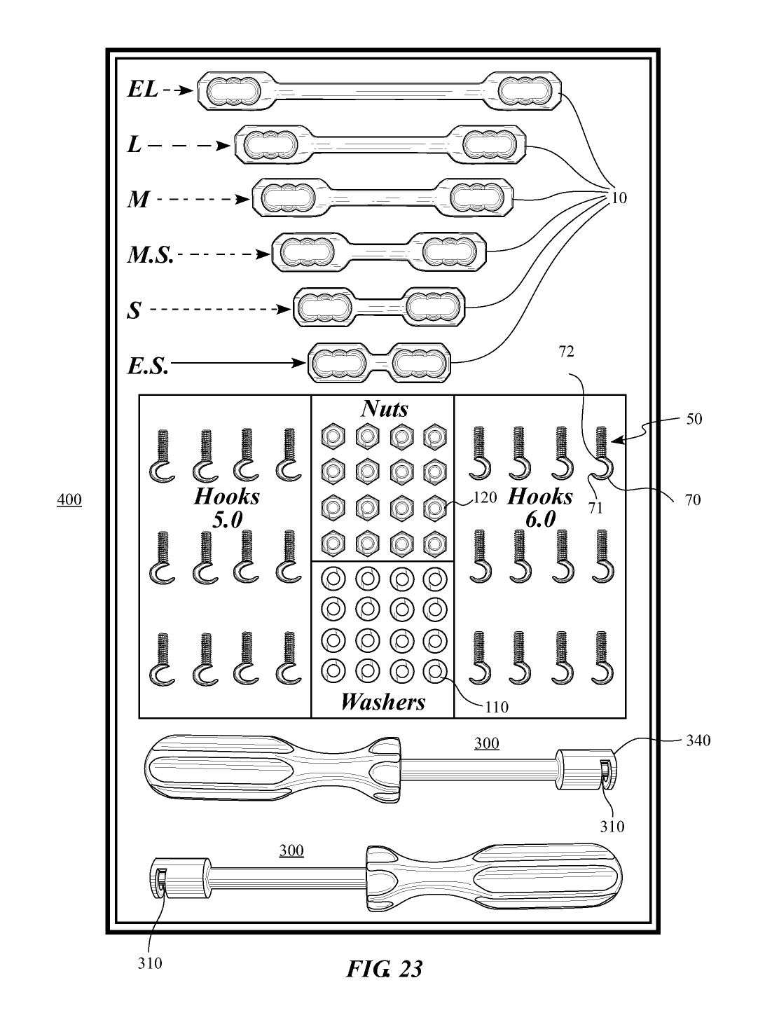

Turning to the issue of stability of the engagement between spinal rod (80) and crosslink element (10), an improved grip of the hook (50) on the rod (80) can be obtained by way of a particular configuration of the hook (70). A first kind of configuration relates to the shape of the hook (70). In particular, reference can be made, for example, to FIG. 23 where it can be seen that hook (70) is C-shaped and the bottom end (71) of the hook (70) is shifted, in a horizontal direction, with respect to a top end (72) of the hook, thus providing a total effective length of the attachment of the hook (70) to the rod (80) of more than half circumference of the rod (80). A second kind of configuration concerns the treatment of the internal surface of the hook (50), i.e. the surface contacting the rod (80), as already mentioned above with reference to FIGS. 9 and 10. The configuration of FIGS. 9 and 10 allows better friction between the hook (50) and the rod (80).

A further feature of the crosslink element (10) is its bendability. Ability of being bent non only before but also during the surgical procedure allows the surgeon to optimally shape the crosslink element taking into account the various differences and space availability in the proximity of each patient's spinal area. For example, utmost care must be exercised to avoid injury to the spinal sack (dura mater).

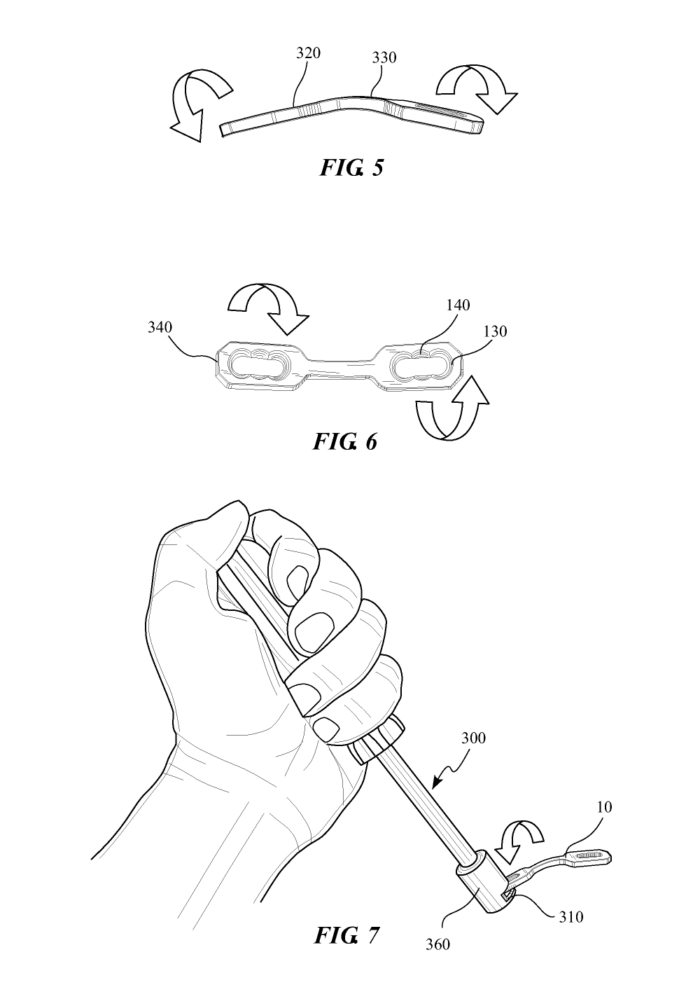

The crosslink element (10) according to the present disclosure is bendable both in a dorsal-ventral direction (bottom-top) and in a cordal-cephalad (lateral) direction. FIG. 5 shows a perspective view indicating bending of the crosslink element in the dorsal-ventral direction. Bendability in the dorsal-ventral direction allows the surgeon to avoid contact with the dural sack under the crosslink element. FIG. 6 shows a perspective view indicating bending of the crosslink element in the cordal-cephalad direction. Bendability in the cordal-cephalad direction provides the surgeon with an additional capability of adapting implantation of the crosslink element to conditions where the relative shape and distance of the spinal rods would not otherwise allow implantation of the crosslink element. Bendability also allows, as previously discussed, a better adaptation to the overall anatomical presentation of a patient.

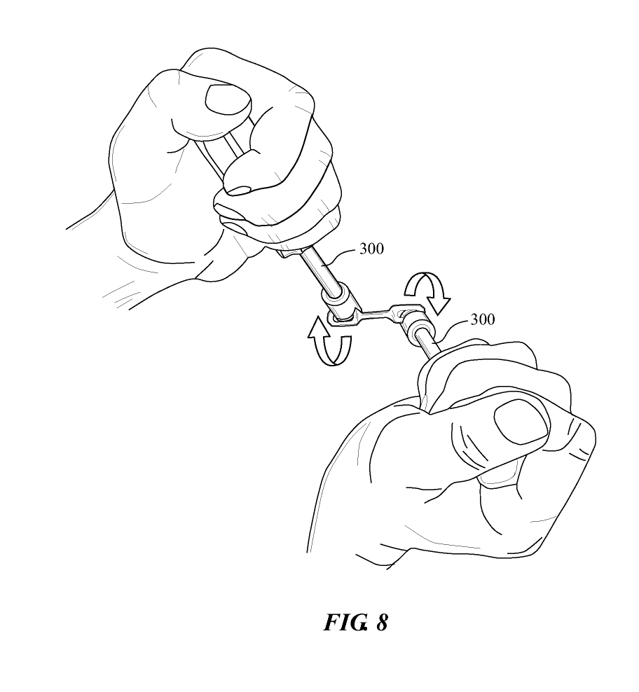

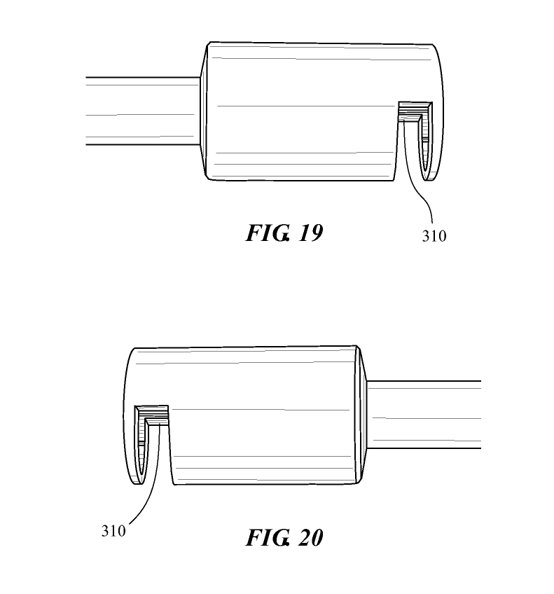

As mentioned above, the crosslink element can be bent also during operation, in its implanted or partially implanted (e.g., secured on a single rod only) condition. In order to do so, a plate bender like bender (300) can be used, as shown in FIGS. 7, 8 and 19-23. Bender (300) comprises a slit (310) where any portion of the crosslink element (10) can be fitted. See, for example, FIGS. 19 and 20. In particular, both a central portion and an end portion of the crosslink element (10) can be fitted. By way of example, two benders (300) can be used to curve crosslink (10) in correspondence of regions (320), (330) (see, e.g., FIG. 5) by locating each region in correspondence of the slit (310) of a corresponding bender (300) and bend the crosslink (10) in a dorsal-ventral direction. On the other hand, if the surgeon's intention is that of bending the crosslink (10) in a cordal-cephalad direction, bender (300) can be used in correspondence of region (340), as also shown in FIG. 7. Additionally, FIG. 8 shows one example where the crosslink element (10) can be twisted with two benders (300).

According to an embodiment of the present disclosure, the distance between the slit (310) and a bottom end (340) of the bender (300) is conveniently reduced to a minimum, in order to avoid possible contact between the bender (300) and the patient's dural sack when performing the bending operation during surgery. See, for example, FIGS. 19 and 20. A possible length of the slit (310) can be half of the circumference of the bottom portion (360) of the bender (300). Applicant has found that such length represents an optimum compromise between structural stability of the bottom portion (360) of the bender (300) and capability of gripping a region as large as possible on the crosslink element (10).

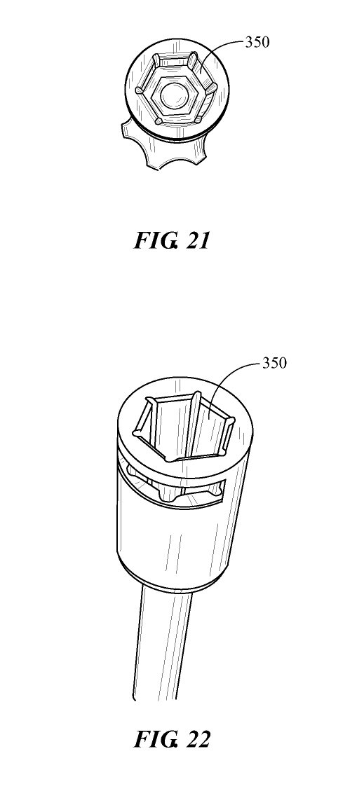

Bender (300) can also engage nut (120) on the hook (50) by way of a nut driver portion (350), shown in FIGS. 21 and 22. Therefore, bender (300) is a multipurpose tool that can be used in conjunction with the crosslink element (10) to bend and engage the crosslink element and to secure the nut and bolts. If desired, a connector, to be inserted in nut driver portion (350), can be added to the bender (300) to provide Philips head and/or screwdriver capabilities. In this respect, the internal surface of nut driver portion (350) can be beveled, if desired, as shown in FIGS. 21 and 22.

Crosslink elements (10) in according to the present disclosure can be provided in a variety of different lengths to allow the surgeon to select the appropriate length according to the section of the spine involved in the surgical procedure. By way of example, six sizes of crosslink elements can be provided. Given the presence of the oval insets around the slots, for each size five different distances between the hooks can be obtained. By way of example, if, for a particular crosslink element, three insets per slot are provided, each distanced 5 mm from the other, and the maximum distance between the hooks (outermost inset to outermost inset) is 55 mm, the following five lengths (in terms of distance between hooks) can be obtained: 55 mm (both hooks at outermost insets), 50 mm (one hook at outermost inset and another hook at middle inset), 45 mm (both hooks at middle insets, or one hook at outermost inset and another hook at innermost inset), 40 mm (one hook at innermost inset and another hook at middle inset), and 35 mm (both hooks at innermost insets). Therefore, in accordance with this example, 30 different lengths are available to the surgeon when operating on the patient.

FIG. 23 shows, by way of example, six different lengths (EL, L, M, MS, S, ES) of the crosslink element. Also shown in FIG. 23 is a possible arrangement, as a kit of parts, of the various parts or components to be used by a surgeon during a surgical procedure. As shown in FIG. 23, an arrangement (400) can contain: 1) multiple crosslink elements (10); 2) multiple hooks (70) having, for example, hook portions with different diameters (e.g., 5 mm and 6 mm) in order to be able to engage with transversal rods (80) having different diameters; 3) multiple washers (110); 4) multiple nuts (120); and 5) one or more benders (300). Such arrangement can be located inside a box to be placed near the surgeon during the procedure.

Various materials, usually metals or metal alloys, can be used to fabricate the crosslink element and the bender. By way of example, the crosslink element can be made of titanium, cobalt chrome alloy, stainless steel, silicon carbide and so on.

If desired, the handle of the benders (300) can have a T-bar shape. Moreover, the handle can be detachable or interchangeable and be made of materials such as metal, rubber, plastic, silicon carbide, and so on.

The examples set forth above are provided to give those of ordinary skill in the art a complete disclosure and description of how to make and use the embodiments of the crosslink element and bender for spine surgery procedures of the disclosure, and are not intended to limit the scope of what the inventors regard as their disclosure. Modifications of the above-described modes for carrying out the disclosure may be used by persons of skill in the art, and are intended to be within the scope of the following claims. All patents and publications mentioned in the specification may be indicative of the levels of skill of those skilled in the art to which the disclosure pertains. All references cited in this disclosure are incorporated by reference to the same extent as if each reference had been incorporated by reference in its entirety individually.

It is to be understood that the disclosure is not limited to particular methods or systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

A number of embodiments of the disclosure have been described. Nevertheless, it will be understood that various modifications may be made without departing from the spirit and scope of the present disclosure. Accordingly, other embodiments are within the scope of the following claims.

1. A substantially flat shaped single piece crosslink element for spine surgery procedures, comprising a central region and end regions, the end regions being wider than the central region, each end region containing a slot adapted to house a hook, wherein the end regions comprise insets provided on one face of the crosslink element in correspondence of borders of the slots.

2. The crosslink element of claim 1, wherein the insets are a plurality of sets of insets.

3. The crosslink element of claim 2, wherein each set of insets comprises a plurality of curved grooves of decreasing diameter in a direction towards the slot.

4. The crosslink element of claim 1, wherein the insets are oval insets.

5. The crosslink element of claim 1, the crosslink element being bendable in a top-bottom direction, thus allowing configurations of the crosslink element where the central region is raised with respect to the end regions.

6. The crosslink element of claim 1, the crosslink element being bendable in a lateral direction, thus allowing configurations of the crosslink element where at least one of the end regions is laterally shifted with respect to the central region.

7. The crosslink element of claim 1, the crosslink element being twistable in a lateral direction, thus allowing configurations of the crosslink element where at least one of the end regions is twisted with respect to the central region.

8. An arrangement comprising the crosslink element of claim 1 and a hook arranged in one of the slots of the crosslink element, the hook forming a separate piece and being adapted to be fixed to the crosslink element.

9. The arrangement of claim 8, wherein the hook comprises a stem portion and a hook portion.

10. The arrangement of claim 9, wherein the stem portion is a threaded stem portion.

11. The arrangement of claim 9, wherein the hook portion comprises an internal surface, the internal surface having an irregular configuration.

12. The arrangement of claim 11, wherein the internal surface comprises a plurality of grooves.

13. The arrangement of claim 11, wherein the internal surface comprises a plurality of beads.

14. The arrangement of claim 11, wherein the internal surface has an irregular configuration only in correspondence of a region of the internal surface proximal to the stem portion.

15. The arrangement of claim 9, wherein the hook portion is adapted to face towards the central region.

16. The arrangement of claim 9, wherein the hook portion is adapted to face outside the central region.

17. The arrangement of claim 8, wherein the hook is provided with a translational degree of freedom and three rotational degrees of freedom.

18. The arrangement of claim 8, wherein each inset in correspondence of a slot defines a position of the hook inside the slot.

19. An arrangement comprising the crosslink element of claim 1 and two hooks, each hook arranged in a respective slot of the crosslink element, the hooks forming separate pieces and being adapted to be independently fixed to the crosslink element.

20. The arrangement of claim 19, wherein each hook comprises a stem portion and a hook portion.

21. The arrangement of claim 20, wherein the stem portion is a threaded stem portion.

22. The arrangement of claim 20, wherein the hook portion comprises an internal surface, the internal surface having an irregular configuration.

23. The arrangement of claim 22, wherein the internal surface comprises a plurality of grooves.

24. The arrangement of claim 22, wherein the internal surface comprises a plurality of beads.

25. The arrangement of claim 22, wherein the internal surface has an irregular configuration only in correspondence of a region of the internal surface proximal to the stem portion.

26. The arrangement of claim 20, wherein both hook portions face towards the central region.

27. The arrangement of claim 20, wherein one hook portion faces towards the central region and the other hook portion faces outside the central region.

28. The arrangement of claim 20, wherein both hook portions face outside the central region.

29. The arrangement of claim 19, wherein each hook is independently provided with a translational degree of freedom and three rotational degrees of freedom.

30. The arrangement of claim 19, wherein each inset in correspondence of a slot defines a position of the hook inside the slot, thus allowing differences distances between the hooks to be obtained.

31. The arrangement of claim 29, wherein the rotational degrees of freedom define a plurality of distances between the hooks in addition to distances between the hooks defined by position of the hooks in the insets.

32. A spinal H-construct or parallelogram, comprising transversal rods and one or more crosslink elements according to claim 1.

33. A spinal H-construct or parallelogram, comprising transversal rods and one or more arrangements according to claim 8.

34. A spinal H-construct or parallelogram, comprising transversal rods and one or more crosslink elements according to claim 19.

35. A kit of parts, comprising a combination of crosslink elements according to claim 1, the combination comprising crosslink elements of different lengths.

36. The kit of parts of claim 35, wherein the different lengths are six different lengths.

37. A hook for surgical procedures comprising a stem portion and a hook portion, the hook portion adapted to be housed in a slot of the crosslink element of claim 1.

38. The hook of claim 37, wherein the stem portion is a threaded stem portion.

39. The hook of claim 37, wherein the hook portion comprises an internal surface, the internal surface having an irregular configuration.

40. The hook of claim 39, wherein the internal surface comprises a plurality of grooves.

41. The hook of claim 39, wherein the internal surface comprises a plurality of beads.

42. The hook of claim 39, wherein the internal surface has an irregular configuration only in correspondence of a region of the internal surface proximal to the stem portion.

43. The hook of claim 37, wherein the hook portion is C-shaped.

44. The hook of claim 43, wherein the hook portion comprises a bottom end and a top end, the bottom end being shifted, in a horizontal direction, with respect to the top end.

45. A surgical bender comprising a bending portion, wherein the bending portion is provided with a slit adapted to host the central region or one of the end regions of the crosslink element of claim 1.

46. The surgical bender of claim 45, further comprising the crosslink element of claim 1, the crosslink element located in the slit of the surgical bender.

47. The surgical bender of claim 45, wherein a distance between the slit and a bottom end of the bender is reduced to a minimum, to avoid contact between the surgical bender and a patient's dural sack when performing a bending operation during surgery.

48. The surgical bender of claim 45, wherein the bending portion has a transversal length, and wherein the slit has a length that is half the transversal length of the bending portion.

49. The surgical bender of claim 45, further comprising a nut driver portion, the surgical bender being a dual purpose bender.

50. The surgical bender of claim 49, further comprising a connector attached to the nut driver portion.

51. A combination of surgical benders comprising two surgical benders according to claim 45.

52. The combination of claim 51, further comprising the crosslink element of claim 1, the crosslink element located in corresponding slits of the two surgical benders.

53. A kit of parts, comprising a combination of crosslink elements according to claim 1, the combination comprising crosslink elements of different lengths, and one or more benders according to claim 45.
